(19) United States
(12) Patent Application Publication
(10) Pub. No.: US 2008/0248015 A1
(43) Pub. Date: Oct. 9, 2008

(54) METHODS AND KITS FOR CO-ADMINISTRATION OF NUTRITIONAL SUPPLEMENTS

(76) Inventors: John A. Giordano, West Orange, NJ (US); Charles J. Balzer, Lavalette, NJ (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/140,895

(22) Filed: Jun. 17, 2008

Related U.S. Application Data
(63) Continuation of application No. 11/034,105, filed on Jan. 13, 2005, now abandoned.

(51) Int. Cl. A61K 31/714; A61K 31/525; A61K 31/51; A61K 31/4415; A61K 31/455; A61K 31/202; A61P 9/00

(52) U.S. Cl. 424/94.1; 514/251; 514/276; 514/350; 514/356; 514/52; 514/560

(57) ABSTRACT

The present invention relates to methods of co-administration of various vitamin and mineral compositions, and in a specific embodiment, comprise co-administering one composition comprising vitamin B₆, vitamin B₁₂, folic acid, and CoQ₁₀ and a second composition comprising omega-3 fatty acids for nutritional supplementation in order to prevent, treat and/or alleviate the occurrence or negative effects of cardiovascular disease; and kits provided for co-administration of various vitamin and mineral compositions, and in a specific embodiment, comprise one composition comprising vitamin B₁₂, folic acid, and CoQ₁₀ and a second composition comprising omega-3 fatty acids for nutritional supplementation in order to prevent, treat and/or alleviate the occurrence or negative effects of cardiovascular disease.

### FIELD OF THE INVENTION

The present invention relates to methods of co-administration of various vitamin and mineral compositions and kits provided for co-administration of these compositions for nutritional supplementation in order to prevent, treat and/or alleviate the occurrence or negative effects of cardiovascular disease including congestive heart failure, ischemic heart disease, cardiomyopathy, hypertension, coronary artery disease, high blood pressure, arrhythmia, and thrombogenicity.

### BACKGROUND OF THE INVENTION

Cardiovascular disease continues to be the number one cause of death for both men and women in the United States. Statistics Related to Heart Disease, available at www.health.uab.edu/show.asp?durki=39661 (last visited 1 Dec. 2004). Correlated risk factors for the incidence of cardiovascular disease include blood level elevations of both cholesterol and homocysteine. Id. Recent developments in nutritional research suggest that nutritional supplementation with specific vitamins and minerals, as an adjunct to proper diet, exercise and medical care, can aid in preventing, treating and/or alleviating the occurrence or negative effects of this disease.

Nutrition plays a critical role in maintaining good health, and nutritional supplementation serves a vital role in protecting against poor nutrition and disease. For example, recent research has shown that vitamins and minerals help to prevent, treat and/or alleviate the occurrence or negative effects of cardiovascular disease. While supplementation with certain vitamins and minerals protects against the onset of these diseases, other vitamins and minerals have been found to inhibit the beneficial effects of these certain vitamins and minerals. Specifically, vitamin B₆, vitamin B₁₂, folic acid, coenzyme Q₁₀ (“CoQ₁₀”), and omega-3 fatty acids play integral roles in physiological mechanisms that serve to prevent, treat and/or alleviate the occurrence or negative effects of cardiovascular disease. Supplementation with other vitamins and minerals may inhibit the beneficial effects of vitamin B₆, vitamin B₁₂, folic acid. CoQ₁₀, and omega-3 fatty acids. Thus, choosing or administering a nutritional supplement, it is essential to understand the physiological needs and risks of individual patients and population groups and the interactions between various vitamins and minerals.

### SUMMARY OF THE INVENTION

The present invention provides methods of co-administering compositions and kits comprising compositions for both prophylactic and therapeutic nutritional supplementation. Specifically, the present invention includes vitamins and minerals that prevent, treat and/or alleviate the occurrence or negative effects of cardiovascular disease. The present invention also may be formulated to exclude vitamins and minerals known to inhibit the beneficial effects of the included vitamins and minerals.

The present invention includes methods of co-administering the compositions of the invention to patients, in any order, to prevent, treat and/or alleviate the occurrence or negative effects of cardiovascular disease including congestive heart failure, ischemic heart disease, cardiomyopathy, hypertension, coronary artery disease, high blood pressure, arrhythmia, and thrombogenicity.

In one embodiment of the present invention, the methods may utilize compositions in a swallowable form. In another embodiment, the methods may utilize compositions substantially free of one or more of other added vitamins, added minerals, and added coenzymes. In another embodiment, the methods may utilize compositions comprising pharmaceutically acceptable carriers, such as one or more of binders, diluents, lubricants, glidants, colorants, emulsifiers, disintegrants, starches, water, oils, alcohols, preservatives and sugars.

In one embodiment of the present invention the methods may utilize compositions comprising vitamin B6, vitamin B12, folic acid, and CoQ10 and compositions comprising omega-3 fatty acids. In another embodiment, methods of the present invention may include folic acid in the form of vitamin B9, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid. 5,10-methenyl-(6R)-tetrahydrofolic acid, and 5-formimino-(6S)-tetrahydrofolic acid or polyglutamyl derivatives thereof.

In another embodiment, the methods may utilize compositions comprising about 15 mg to about 45 mg vitamin B6; about 0.9 mg to about 2.7 mg vitamin B12; about 1.25 mg to about 3.75 mg folic acid; and about 50 mg to about 150 mg CoQ10.

In yet another embodiment, the methods may utilize compositions comprising about 24 mg to about 36 mg vitamin B6; about 1.44 mg to about 2.16 mg vitamin B12; about 2 mg to about 3 mg folic acid; and about 80 mg to about 120 mg CoQ10.

In another embodiment, the methods may utilize compositions comprising about 27 mg to about 33 mg vitamin B₆; about 1.26 mg to about 1.98 mg vitamin B₁₂; about 2.25 mg to about 2.75 mg folic acid; and about 90 mg to about 110 mg CoQ₁₀.

In another embodiment, the methods may utilize compositions comprising about 30 mg vitamin B₆; about 1.8 mg vitamin B₁₂; about 2.5 mg folic acid; and about 100 mg CoQ₁₀. In one embodiment, the methods may utilize compositions comprising about 50 mg to about 150 mg omega-3 fatty acids. In another embodiment, the methods may utilize compositions comprising about 75 mg to about 125 mg omega-3 fatty acids. In yet another embodiment, the methods may utilize compositions comprising about 90 mg to about 110 mg omega-3 fatty acids. Alternatively, the methods may utilize compositions comprising about 100 mg omega-3 fatty acids.

In another specific embodiment, the methods may utilize compositions comprising omega-3 fatty acids enclosed within a gelatin capsule (commonly known as a “gel-cap”).

In one embodiment, the methods may comprise co-administering to a patient one unit dose of compositions comprising vitamin B₆, vitamin B₁₂, folic acid, and CoQ₁₀ and one unit dose of compositions comprising omega-3 fatty acids, at the same time.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 1 minute following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 2 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 3 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 4 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 5 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 6 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 7 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 8 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 9 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 10 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 11 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 12 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 13 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 14 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 15 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 16 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 17 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 18 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 19 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 20 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 21 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 22 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 23 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 24 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 25 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 26 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 27 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 28 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 29 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 30 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 31 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 32 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 33 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 34 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 35 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 36 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 37 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 38 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 39 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 40 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 41 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 42 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 43 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 44 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 45 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 46 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 47 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 48 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 49 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 50 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 51 minutes following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 52 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 53 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 54 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 55 minutes following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 56 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 57 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 58 minutes following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 59 minutes following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 60 minutes following the administration of the other.

In one embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 2 hours following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 3 hours following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 4 hours following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 5 hours following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 6 hours following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 7 hours following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 8 hours following the administration of the other.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 9 hours following the administration of the other.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 10 hours following the administration of the other.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 11 hours following the administration of the other.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, one administered about 12 hours following the administration of the other.

In one embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once a day.

In an alternative embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once every 2 days.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once every 3 days.

In yet another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once every 4 days.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once every 5 days.

In another specific embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once every 6 days.

In another embodiment, the methods may comprise co-administering to a patient one unit dose of compositions comprising vitamin B₆, vitamin B₁₂, folic acid, and CoQ₁₀ and one unit dose of compositions comprising omega-3 fatty acids, in either order, together once a week.

The present invention includes kits that may be provided to patients to prevent, treat and/or alleviate the occurrence or negative effects of cardiovascular disease including congestive heart failure, ischemic heart disease, cardiomyopathy, hypertension, coronary artery disease, high blood pressure, arrhythmia, and thrombogenicity.

In one embodiment of the present invention, the kits may comprise compositions in a swallowable form. In another embodiment, the kits may comprise compositions substantially free of one or more of other added vitamins, added minerals, and added coenzymes. In another embodiment, the kits may comprise compositions comprising pharmaceutically acceptable carriers, such as one or more of binders, diluents, lubricants, glidants, colorants, emulsifiers, disintegrants, starches, water, oils, alcohols, preservatives and sugars.

In one embodiment of the present invention, the kits may comprise compositions comprising vitamin B₆, vitamin B₁₂, folic acid, and CoQ₁₀ and compositions comprising omega-3 fatty acids packaged for co-administration to a patient, in any order. In another embodiment, the kits may include folic acid in the form of vitamin B₉, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In one embodiment, the kits may comprise compositions comprising about 15 mg to about 45 mg vitamin B₆; about 0.9 mg to about 2.7 mg vitamin B₁₂; about 1.25 mg to about 3.75 mg folic acid; and about 50 mg to about 150 mg CoQ₁₀.

In an alternative embodiment, the kits may comprise compositions comprising about 24 mg to about 36 mg vitamin B₆; about 1.44 mg to about 2.16 mg vitamin B₁₂; about 2 mg to about 3 mg folic acid; and about 80 mg to about 120 mg CoQ₁₀.

In another embodiment, the kits may comprise compositions comprising about 27 mg to about 33 mg vitamin B₆; about 1.26 mg to about 1.98 mg vitamin B₁₂; about 2.25 mg to about 2.75 mg folic acid; and about 90 mg to about 110 mg CoQ₁₀.

In yet another embodiment, the kits may comprise compositions comprising about 30 mg vitamin B₆; about 1.8 mg vitamin B₁₂; about 2.5 mg folic acid; and about 100 mg CoQ₁₀.

In one embodiment, the kits may comprise compositions comprising about 50 mg to about 150 mg omega-3 fatty acids.

In an alternative embodiment, the kits may comprise compositions comprising about 75 mg to about 125 mg omega-3 fatty acids.

In another embodiment, the kits may comprise compositions comprising about 90 mg to about 110 mg omega-3 fatty acids.

In yet another embodiment, the kits may comprise compositions comprising about 100 mg omega-3 fatty acids.

In a specific embodiment, the kits may comprise compositions comprising omega-3 fatty acids enclosed within a gel-cap.

In another embodiment, the kits may be packaged in various forms including bottles and blister packs.

In yet another embodiment, the kits may be packaged in bottles that are sold together; one bottle containing compositions comprising vitamin B₆, vitamin B 2, folic acid, and CoQ₁₀ and one bottle containing compositions comprising omega-3 fatty acids.

In another embodiment, the kits may be packaged in bottles that are sold separately; one bottle containing compositions comprising vitamin B₆, vitamin B₂, folic acid, and CoQ₁₀ and one bottle containing compositions comprising omega-3 fatty acids.

In an alternative embodiment, the kits may be packaged in bottles advertised as more effective if co-administered; one bottle containing compositions comprising vitamin B₆, vitamin B₁₂, folic acid, and CoQ₁₀ and one bottle containing compositions comprising omega-3 fatty acids. The advertisements may consist of internet, print, and product packaging advertisements.

In another embodiment, the kits may be packaged in blister packs that are sold together; one blister pack containing compositions comprising vitamin B₆, vitamin B₁₂, folic acid, and CoQ₁₀ and one blister pack containing compositions comprising omega-3 fatty acids.

In yet another embodiment, the kits may be packaged in one blister pack containing compositions comprising vitamin B₆, vitamin B₁₂, folic acid, and CoQ₁₀ and containing compositions comprising omega-3 fatty acids paired together per unit dose.

In another embodiment, the kits may be packaged in blister packs that are sold separately; one blister pack containing compositions comprising vitamin B₆, vitamin B₁₂, folic acid, and CoQ₁₀ and one blister pack containing compositions comprising omega-3 fatty acids.

In yet another embodiment, the kits may be packaged in blister packs advertised as more effective if co-administered; one blister pack containing compositions comprising vitamin B₆, vitamin B₁₂, folic acid, and CoQ₁₀ and one blister pack containing compositions comprising omega-3 fatty acids. The advertisements may consist of internet, print, and product packaging advertisements.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, and excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “subject,” as used herein, comprises any and all organisms and includes the term “patient.” “Subject” may refer to a human or any other animal.

The phrase “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “swallowable form” refers to any compositions that do not readily dissolve when placed in the mouth and may be swallowed whole without any chewing or discomfort. In one embodiment, may have a shape containing no sharp edges and a smooth, uniform and substantially bubble free outer coating.

The phrase “co-administration” refers to administration of two compositions to a patient within a certain desired time.

As stated earlier, cardiovascular disease is the number one cause of death for adults in the United States. Recent developments in nutritional research suggest that nutritional supplementation with certain vitamins and minerals, as an adjunct to proper diet, exercise and medical care, can aid in preventing, treating and/or alleviating the occurrence or negative effects of these diseases.

The methods and kits of the present invention provide means to optimize good health by utilizing vitamin and mineral combinations that specifically aim to prevent, treat and/or alleviate the occurrence or negative effects of cardiovascular disease. The methods and kits of the present invention may be administered to or directed to a subject such as a human or any other organism. Each of the added vitamins and minerals that can be included in the present invention including vitamin B₆ vitamin B₁₂, folic acid. CoQ₁₀, and omega-3 fatty acids, plays a specific role in preventing, treating and/or alleviating the occurrence or negative effects of cardiovascular disease. In a specific embodiment, vitamins, minerals, and coenzymes that inhibit these compounds' beneficial effects, may be specifically excluded from the methods and kits of the present invention. Further, in another specific embodiment, other added vitamins and/or minerals and/or coenzymes can be excluded.

The methods and kits of the present invention comprise compositions that may be co-administered. Co-administration of the compositions of the present invention and/or patient compliance may be found to achieve an improved clinical outcome preventing, treating and/or alleviating the occurrence or negative effects of cardiovascular disease.

Vitamin B₆ and vitamin B₁₂ are water-soluble nutrients that generally are not stored in the body. These vitamins play a variety of roles within the body. They are included in the methods and kits of the present invention due to their roles in preventing, treating and/or alleviating the occurrence or negative effects of cardiovascular disease.

Vitamin B₆, vitamin B₁₂, and folic acid help prevent, treat and/or alleviate the occurrence or negative effects of cardiovascular disease through their involvement in the metabolism and breakdown of homocysteine. Homocysteine is an intermediary product produced by metabolism of the amino acid methionine. Elevated levels of homocysteine have been correlated to an increased risk of cardiovascular disease. Maxwell, Suppl 1 BASIC RES. CARDOL. 165-71 (2003). Elevated levels of homocysteine may lead to increased risk of cardiovascular disease due to this metabolite's numerous deleterious effects on the vascular system including impairing endothelial function, inducing thrombosis, and increasing oxidant stress. Schlaich, 153(2) ATHEROSCLER. 383-89 (2000); Hanratty, 85(3) HEART 326-30 (2001). Efficient breakdown of this intermediary product is necessary to avoid these deleterious effects on the vascular system, and the metabolic breakdown pathways of homocysteine require vitamins B₆, B₁₂ and folic acid. Thus, optimizing the levels of these vitamins has cardio-protective effects by promoting the efficient degradation of homocysteine. Haynes, 16(5) CARDIOVASC. DRUGS THER. 391-9 (2002).

Folic acid is essential for the formation of red and white blood cells within bone marrow and also plays a role in heme formation. National Research Council, RECOMMENDED DIETARY ALLOWANCES 10^{th} ed., 150 (1989). Folic acid in its active form, tetrahydrofolate, is a coenzyme that is involved in the transfer of methyl groups and it plays a role in DNA synthesis, purine synthesis, and amino acid synthesis, such as the conversion of glycine to serine and the transformation of homocysteine to methionine. The activation of folic acid requires a vitamin B₁₂-dependent transmethylation and vitamin B₁₂ is also necessary for folic acid delivery to tissues. Id.

Folic acid also may have additional physiological effects beyond its role in homocysteine breakdown that protect against cardiovascular disease. Bailey, 133(6) J. NUTR. 1961S-68S (2003); Doshi, 41(11) CLIN. CHEM. LAB. MED. 1505-12 (2003); Haynes, supra. For example, folic acid improves the levels and functioning of the health promoting, endothelial-derived compound nitric oxide (NO). Das, 19(7-8) NUTR. 686-92 (2003). Folic acid creates this effect by enhancing the activity of the enzyme nitric oxide synthase, stimulating endogenous tetrahydrobiopterin, and inhibiting generation of intracellular superoxide. All of these actions enhance the half-life of NO thus creating cardioprotective effects. Lucock, 71 MOL. GENET. METAB. 121-38 (2000).

In one embodiment, the methods and kits of the present invention may include vitamin B₆ in amounts ranging from about 15 mg to about 45 mg. In another embodiment, the methods and kits of the present invention may include vitamin B₆ in amounts ranging from about 24 mg to about 36 mg. In yet another embodiment, the methods and kits of the present invention may include vitamin B₆ in amounts ranging from about 27 mg to about 33 mg. In a specific embodiment, the methods and kits of the present invention may include vitamin B₆ in an amount of about 30 mg.

Vitamin B₁₂ also may be included in the methods and kits of the present invention. In one embodiment of the present invention, vitamin B₁₂ may be included in amounts ranging from about 0.9 mg to about 2.7 mg. In another embodiment, vitamin B₁₂ may be included in amounts ranging from about 1.44 mg to about 2.16 mg. In yet another embodiment, vitamin B₁₂ may be included in amounts ranging from about 1.26 mg to about 1.98 mg. In a specific embodiment of the present invention, vitamin B₁₂ may be included in an amount of about 1.8 mg.

Folic acid also may be included in the methods and kits of the present invention. In one embodiment of the present invention, folic acid may be included in amounts ranging from about 1.25 mg to about 3.75 mg. In another embodiment of the present invention folic acid may be included in amounts ranging from about 2 mg to about 3 mg. In yet another embodiment, folic acid may be included in amounts ranging from about 2.25 mg to about 2.75 mg. In a specific embodiment of the present invention, folic acid may be included in an amount of about 2.5 mg.

CoQ₁₀ belongs to the ubiquinone family of substances. CoQ₁₀ is a fat soluble, vitamin-like substance that is an essential cofactor in the mitochondrial electron transport chain and thus is vital in the production of Adenosine-Tri-Phosphate (ATP). CoQ₁₀ is biosynthesized in the body and shares a common synthetic pathway with cholesterol. Sarter, 16(4) J CARDIOVASC. NURS. 9-20 (2002).

Early studies have documented a deficiency of CoQ₁₀ in patients with a variety of cardiovascular disorders including, but not limited to, congestive heart failure, ischemic heart disease, cardiomyopathy, and hypertension. Id. Studies have indicated that administration of CoQ₁₀ provides a therapeutic benefit to patients with cardiovascular disease. CoQ₁₀ has been shown to significantly reduce blood pressure in patients with hypertension. Id. Administration of CoQ₁₀ to patients with congestive heart failure has been shown to improve myocardial metabolic function, reduce blood pressure, and reduce myocardial thickness. Id. Further, CoQ₁₀ enhances both the magnitude and rate of clinical improvement in patients with ischemic heart disease. Langsjoen et al., 9(24) BIOFACTORS. 273-284 (1999). Additionally, studies have suggested CoQ₁₀ may be a benefit in treating patients with cardiomyopathy. Patients treated with CoQ₁₀ showed significant improvement in cardiac function including ejection fraction and cardiac output. J CARDIOVASC. NURS. at 9.

Nutritional supplementation with CoQ₁₀ can help prevent, treat and/or alleviate the occurrence or negative effects of cardiovascular disease. In one embodiment, the methods and kits of the present invention may include CoQ₁₀ in amounts ranging from about 50 mg to about 150 mg. In another embodiment, the methods and kits of the present invention may include CoQ₁₀ in amounts ranging from about 80 mg to about 120 mg. In yet another embodiment, the methods and kits of the present invention may include CoQ₁₀ in amounts ranging from about 90 mg to about 110 mg. In a specific embodiment, the methods and kits of the present invention may include CoQ₁₀ in an amount of about 100 mg.

Omega-3 fatty acids, also known as (n−3) fatty acids, are long-chain polyunsaturated fatty acids. Holub, 166 CANADIAN MEDICAL ASSOCIATION JOURNAL 608-615 (2002), available at http://www.mercola.com/2002/mar/27/omega3_fats.htm. Omega-3 fatty acids are characterized as Essential Fatty Acids (EFAs). EFAs are required for the formation of a variety of eicosanoids, including prostaglandins, thromboxanes, prostacyclins, and leukotrienes. THE MERCK MANUAL, 17th Ed., 2-3, 32-33 (1999). EFAs are needed for many physiologic processes including maintaining the integrity of the skin, maintaining the structure of cell membranes, and synthesizing prostaglandins and leukotrienes. Id.

Omega-3 fatty acids are important in cardiovascular care. Increasing the intake of omega-3 fatty acids through diet and supplementation results in a corresponding increase of these omega-3 fatty acids in tissue, cellular lipids, and circulatory lipids along with a simultaneous reduction in omega-6 fatty acids. See Holub supra. This fatty acid shift alters the physicochemical properties of cell membranes and their functioning, modifies cell signaling, gene expression and biosynthetic processes and eicosanoid formations. Id. This shift leads to beneficial cardiovascular effects. Benefits include decreased platelet adhesiveness and aggregation, overall reduction in thrombogenicity, antiatherogenic effects, lowered levels of blood triglycerides (elevated levels of triglycerides are linked with a progressively increased risk of ischemic heart disease), lowered blood pressure levels, reduction in arrhythmia, and a decreased the risk of coronary artery disease. Kirs-Etherton, 106 CIRCULATION 2747-2757 (2002); See also THE MERCK MANUAL at 3; Holub supra.

Nutritional supplementation with omega-3 fatty acids can help prevent, treat and/or alleviate the occurrence or negative effects of cardiovascular disease. In one embodiment, the methods and kits of the present invention may include omega-3 fatty acids in amounts ranging from about 50 mg to about 150 mg. In another embodiment, the methods and kits of the present invention may include omega-3 fatty acids in amounts ranging from about 80 mg to about 120 mg. In yet another embodiment, the methods and kits of the present invention may include omega-3 fatty acids in amounts ranging from about 90 mg to about 110 mg. In a specific embodiment, the methods and kits of the present invention may include omega-3 fatty acids in an amount of about 100 mg.

The active ingredients are available from numerous commercial sources, and in several active forms or salts thereof, known to those of ordinary skill in the art. Hence, the methods and kits of the present invention are not limited to comprising or using any particular form of the vitamin or mineral ingredient described herein.

Nutrition is a constantly evolving health science. Nearly as proliferative as research findings correlating nutrients and disease prevention are findings demonstrating that supplementation with some nutrients can be counter-productive to the health needs of specific populations. In a specific embodiment, the methods and kits of the present invention may be substantially free of other added vitamins, minerals, and coenzymes.

The compositions of the present invention are preferably administered in amounts to patients that provide the supplementation required to alleviate the vitamin deficiencies associated with cardiovascular disease. An exemplary dosage of the compositions of the present invention may consist of one or more caplets or gel-caps for human oral consumption. If more than one caplet or gel-cap is used, each individual caplet or gel-cap may be identical to the other caplets or gel-caps, or each may contain only some of the ingredients of the composition, so that the combination of the different caplets or gel-caps comprises a composition of the present invention.

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention, the swallowable compositions may have a shape containing no sharp edges and a smooth, uniform and substantially bubble free outer coating.

A specific embodiment of the present invention may comprise swallowable compositions comprising vitamin B₆, vitamin B₁₂, folic acid, and CoQ₁₀ in caplet form. Another specific embodiment of the present invention may comprise swallowable compositions comprising omega-3 fatty acids enclosed within a gel-cap.

In another specific embodiment, the swallowable compositions of the present invention may be in the form of gel-caps. Gel-caps consist of a filler comprising one or more pharmaceutically active materials dissolved or dispersed in an appropriate liquid vehicle encapsulated in a gelatin shell generally comprising gelatin together with a plasticizer such as glycerin or sorbitol. The filler material may comprise, for example, polyethylene glycols. Gel-caps are well known to those of ordinary skill in the art. See for example, U.S. Pat. Nos. 4,780,316; 5,419,916; 5,641,512; and 6,589,536.

To prepare the swallowable compositions in caplet form, each of the active ingredients may be combined in intimate admixture with a suitable carrier according to conventional compounding techniques. In a specific embodiment of swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, e.g., WADE & WALLER, HANDBOOK OF PHARMACEUTICAL EXCIPIENTS (2^{nd} ed. 1994).

Disintegrants also may be included in the compositions of the present invention in order to facilitate dissolution. Disintegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include by way of example and without limitation, starches such as corn starch, potato starch, pre-gelatinized and modified starches thereof, cellulosic agents such as Ac-di-sol, montmorrilonite clays, cross-linked PVP, sweeteners, bentonite, microcrystalline cellulose, croscarmellose sodium, alginates, sodium starch glycolate, gums such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents such as hydroxymethylcellulose, hydroxypropylcellulose and hydroxypropylmethylcellulose.

Finally, dissolution of the compositions may be facilitated by including relatively small particles sizes of the ingredients used.

In addition to those described above, any appropriate fillers and excipients may be utilized in preparing the swallowable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents such as grape skin extract, beet red powder, beta carotene, annato, carmine, turmeric, paprika, and others known to those of ordinary skill in the art. It is recognized that no colorant is required in the nutritional supplement compositions described herein.

If desired, compositions may be sugar coated or enteric coated by standard techniques.

The swallowable compositions of the present invention may be prepared using conventional methods and materials known in the pharmaceutical art. For example, U.S. Pat. Nos. 5,215,754 and 4,374,082 relate to methods for preparing swallowable compositions. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in WADE & WALLER, supra. All active ingredients, fillers and excipients are commercially available from companies such as Aldrich Chemical Co., FMC Corp, Bayer, BASF, Alexi Fres, Witco, Mallinckrodt, Rhodia, ISP, and others.

A specific embodiment of the present invention may comprise swallowable compositions packaged in blister packs. Blister packs as packaging for swallowable compositions are well known to those of ordinary skill in the art. Blister packs may be made of a transparent plastic sheet which as been formed to carry a matrix of depression or blisters. One or more swallowable compositions are received in each depression or blister. A foil or plastic backing is then adhered across the plane of the sheet sealing the swallowable compositions in their respective blisters. Examples of materials used for the blister packs include, but are not limited to, aluminum, paper, polyester, PVC, and polypropylene. Alternative materials are known to those of ordinary skill in the art. To remove a swallowable composition, the depression material is pressed in and the composition is pushed through the backing material. Multiple blister packs may be placed in an outer package, often a box or carton for sale and distribution.

Another specific embodiment of the present invention may comprise swallowable compositions packaged in bottles. The bottle may be glass or plastic in form with a pop or screw top cap. Bottle packaging for compositions in swallowable form are well known to those of ordinary skill in the art.

Additionally, the unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A first composition of the following formulation was prepared in caplet form by standard methods known to those skilled in the art:

| | | |
|---|---|---|
| Vitamin B₆ | 30 | mg |
| Vitamin B₁₂ | 1.8 | mg |
| Folic acid | 2.5 | mg |
| CoQ₁₀ | 100 | mg |

A second composition of the following formulation was prepared in gel-cap form by standard methods known to those skilled in the art:

| | |
|---|---|
| Omega-3 fatty acids | 100 mg |

### Example 2

A study is undertaken to evaluate the effectiveness of the compositions of the present invention in the treatment of patients. The objective of the study is to determine whether oral co-administration of the compositions results in an improvement of the nutritional status with regard to specific vitamins and minerals contained in the co-administered compositions.

A double-blind, placebo controlled study is conducted over a six-month period. A total of 120 subjects, aged 30-45 years, are chosen for the study. An initial assessment of the nutritional status of each subject is conducted. Vitamin B₆ is measured by a radioenzymatic assay method wherein serum is incubated with apoenzyme tyrosine-decarboxylase, C₁₄ labeled tyrosine is added to start the enzymatic reaction which is stopped with HCl. Subsequently the free C₁₄-labelled CO₂ is adsorbed by a KOH impregnated filtering paper. The measured C₁₄ activity is directly proportional to the B₆ concentration. Vitamins B₁₂ and folic acid are measured by quantitative radioassay methods using purified intrinsic factor and purified folate binding protein. CoQ₁₀ is measured by using column-switching high performance liquid chromatography (HPLC). Determination of CoQ₁₀ is performed on a reversed-phase analytical column with ultraviolet detection at 275 nm. The mobile phase contains 10% (v/v) isopropanol in methanol at a flow rate of 1.5 m./min. This method allows for the detection of 0.1 microg/ml CoQ₁₀ in plasma (S/N=3). Omega-3 fatty acids are measured and quantified using gas chromatography procedures.

The 120 subjects are separated into 4 separate groups of 30 subjects. In a first group comprising men, and in a second group comprising women, each subject is co-administered one dosage form of the first composition and one dosage form of the second composition as described in Example 1 once a day. In a third group comprising men and a fourth group comprising women, each subject is co-administered one placebo dosage form for each composition once a day. Thus, dosage form co-administration occurs every 24 hours. No other nutritional supplements are taken by the subjects during the assessment period.

An assessment of the nutritional status of each subject is conducted utilizing methods described above at one month intervals for a six month period. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINCAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1, 2, 3, 4, 5 and 6 months, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status of all vitamin and mineral levels measured is observed in the treated subjects over the controls upon completion of the study. Therefore, the study confirms that oral co-administration of the two compositions of the present invention is effective in improving the nutritional status of patients.

While specific embodiments of the present invention have been described, other and further modifications and changes may be made without departing from the spirit of the invention. All further and other modifications and changes are included that come within the scope of the invention as set forth in the claims. The disclosure of all publications cited above are expressly incorporated by reference in their entireties to the same extent as if each were incorporated by reference individually.

1-77. (canceled)

78. A method comprising co-administering a first composition consisting of vitamin B₆ vitamin B₁₂, folic acid, CoQ₁₀ and one or more pharmaceutically acceptable carriers to a patient and a second composition consisting of omega-3 fatty acids and one or more pharmaceutically acceptable carriers to said patient, in any order.

79. The method of claim 78, wherein said folic acid is selected from the group consisting of vitamin B₉, folacin, metafolin, folate, (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid and 5-formimino-(6S)-tetrahydrofolic acid.

80. The method of claim 78, wherein said pharmaceutically acceptable carrier of said first composition is selected from one or more of the group consisting of binders, diluents, lubricants, glidants, colorants, emulsifiers, disintegrants, starches, water, oils, alcohols, preservatives and sugars.

81. The method of claim 78, wherein said vitamin B₆ is present in the range of about 15 mg to about 45 mg.

82. The method of claim 78, wherein said vitamin B₁₂ is present in the range of about 0.9 mg to about 2.7 mg.

83. The method of claim 78, wherein said folic acid is present in the range of about 1.25 mg to about 3.75 mg.

84. The method of claim 78, wherein said CoQ₁₀ is present in the range of about 50 mg to about 150 mg.

85. The method of claim 78, wherein said first composition consists of about 15 mg to about 45 mg vitamin B₆, about 0.9 mg to about 2.7 mg vitamin B₁₂; about 1.25 mg to about 3.75 mg folic acid; and about 50 mg to about 150 mg CoQ₁₀.

86. The method of claim 78, wherein said first composition consists of about 24 mg to about 36 mg vitamin B₆; about 1.44 mg to about 2.16 mg vitamin B₁₂; about 2 mg to about 3 mg folio acid; and about 80 mg to about 120 mg CoQ₁₀.

87. The method of claim 78, wherein said first composition consists of about 27 mg to about 33 mg vitamin 0.13; about 1.26 mg, to about 1.98 mg vitamin B₁₂; about 2.25 mg to about 2.75 mg folic acid; and about 90 mg to about 110 mg CoQ₁₀.

88. The method of claim 87, wherein said vitamin B₆ is present in the amount of about 30 mg.

89. The method of claim 87, wherein said vitamin B₁₂ is present in the amount of about 1.8 mg.

90. The method of claim 87, wherein said folic acid is present in the amount of about 2.5 mg.

91. The method of claim 87, wherein said CoQ₁₀ is present in the amount of about 100 mg.

92. The method of claim 78, wherein said vitamin B₆ is present in the amount of about 30 mg; said vitamin B₁₂ is present in the amount of about 1.8 mg; said folic acid is present in the amount of about 2.5 mg; and said CoQ₁₀ is present in the amount of about 100 mg.

93. The method of claim 78, wherein said first composition is administered to said patient orally.

94. The method of claim 78, wherein said first composition is administered to said patient to treat and/or alleviate the occurrence or negative effects of cardiovascular disease.

95. The method of claim 94, wherein said cardiovascular disease is selected from one or more of the group consisting of congestive heart failure, ischemic heart disease, cardiomyopathy, and hypertension.

96. The method of claim 78, wherein said pharmaceutically acceptable carrier of said second composition is selected from one or more of the group consisting of binders, diluents, lubricants, glidants, colorants, emulsifiers, disintegrants, starches, water, oils, alcohols, preservatives and sugars.

97. The method of claim 78, wherein said omega-3 fatty acids are enclosed within a gel-cap.

98. The method of claim 78, wherein said omega-3 fatty acids are present in the amount of about 50 mg to about 150 mg.

99. The method of claim 78, wherein said omega-3 fatty acids are present in the amount of about 75 mg to about 125 mg.

100. The method of claim 78, wherein said omega-3 fatty acids are present in the amount of about 90 mg to about 110 mg.

101. The method of claim 78, wherein said omega-3 fatty acids are present in the amount of about 100 mg.

102. The method of claim 78, wherein said second composition is administered to said patient orally.

103. The method of claim 78, wherein said second composition is administered to said patient to treat and/or alleviate the occurrence or negative effects of cardiovascular disease.

104. The method of claim 103, wherein said cardiovascular disease is selected from one or more of the group consisting of ischemic heart disease, coronary artery disease, high blood pressure, arrhythmia, and thrombogenicity.

105. A kit comprising a first composition consisting of vitamin B₆, vitamin B₁₂, folic acid, CoQ₁₀ and one or more pharmaceutically acceptable carriers; and a second composition consisting of omega-3 fatty acids and one or more pharmaceutical acceptable carriers wherein said first and said second compositions are packaged for co-administration to a patient, in any order.

106. The kit of claim 105, wherein said folic acid is selected from the group consisting of vitamin B₉, folacin, metafolin, folate, (6S)-tetrahydrofolic acid, 5-methyl-(6S)-tetrahydrofolic acid, 5-formyl-(6S)-tetrahydrofolic acid. 10-formyl-(6R)-tetrahydrofolic acid, 5,10-methylene-(6R)-tetrahydrofolic acid, 5,10-methenyl-(6R)-tetrahydrofolic acid and 5-formimino-(6S)-tetrahydrofolic acid.

107. The kit of claim 105, wherein said pharmaceutically acceptable carrier from said first composition is selected from one or more of the group consisting of binders, diluents, lubricants, glidants, colorants, emulsifiers, disintegrants, starches, water, oils, alcohols, preservatives and sugars.

108. The kit of claim 105, wherein said vitamin B₆ is present in the range of about 15 mg to about 45 mg.

109. The kit of claim 105, wherein said vitamin B₁₂ is present in the range of about 0.9 mg to about 2.7 mg.

110. The kit of claim 105, wherein said folio acid is present in the range of about 1.25 mg to about 3.75 mg.

111. The kit of claim 105, wherein said CoQ₁₀ is present in the range of about 50 mg to about 150 mg.

112. The kit of claim 105, wherein said first composition consists of about 15 mg to about 45 mg vitamin B₆; about 0.9 mg to about 2.7 mg vitamin B₁₂; about 1.25 mg to about 3.75 mg folic acid; and about 50 mg to about 150 mg CoQ₁₀.

113. The kit of claim 105, wherein said first composition consists of about 24 mg to about 36 mg vitamin B₆; about 1.44 mg to about 2.16 mg vitamin B₁₂; about 2 mg to about 3 mg folic acid; and about 80 mg to about 120 mg CoQ₁₀.

114. The kit of claim 105, wherein said first composition consists of about 27 mg to about 33 mg vitamin B₆; about 1.26 mg to about 1.98 mg vitamin B₁₂; about 2.25 mg to about 2.75 mg folic acid; and about 90 mg to about 110 mg CoQ₁₀.

115. The kit of claim 114, wherein said vitamin B₆ is present in the amount of about 30 mg.

116. The kit of claim 114, wherein said vitamin B₁₂ is present in the amount of about 1.8 mg.

117. The kit of claim 114, wherein said folic acid is present in the amount of about 2.5 mg.

118. The kit of claim 114, wherein said CoQ₁₀ is present in the amount of about 100 mg.

119. The kit of claim 105, wherein said vitamin B₆ is present in the amount of about 30 mg; said vitamin B′ is present in the amount of about 1.8 mg; said folic acid is present in the amount of about 2.5 mg; and said CoQ₁₀ is present in the amount of about 100 mg.

120. The kit of claim 105, wherein said first composition is administered to said patient orally.

121. The kit of claim 105, wherein said first composition is administered to said patient to treat and/or alleviate the occurrence or negative effects of cardiovascular disease.

122. The kit of claim 121, wherein said cardiovascular disease is selected from one or more of the group consisting of congestive heart failure, ischemic heart disease, cardiomyopathy, and hypertension.

123. The kit of claim 105, wherein said pharmaceutically acceptable carrier from said second composition is selected from one or more of the group consisting of binders, diluents, lubricants, glidants, colorants, emulsifiers, disintegrants, starches, water, oils, alcohols, preservatives and sugars.

124. The kit of claim 105, wherein said omega-3 fatty acids are enclosed within a gel-cap.

125. The kit of claim 105, wherein said omega-3 fatty acids are present in the amount of about 50 mg to about 150 mg.

126. The kit of claim 105, wherein said omega-3 fatty acids are present in the amount of about 75 mg to about 125 mg.

127. The kit of claim 105, wherein said omega-3 fatty acids are present in the amount of about 90 mg to about 110 mg.

128. The kit of claim 105, wherein said omega-3 fatty acids are present in the amount of about 100 mg.

129. The kit of claim 105, wherein said second composition is administered to said patient orally.

130. The kit of claim 105, wherein said second composition is administered to said patient to treat and/or alleviate the occurrence or negative effects of cardiovascular disease.

131. The kit of claim 130, wherein said cardiovascular disease is selected from one or more of the group consisting of ischemic heart disease, coronary artery disease, high blood pressure, arrhythmia, and thrombogenicity.

132. The kit of claim 105, wherein said packaging is selected from the group consisting of bottles and blister packs.

133. The kit of claim 132, wherein said bottles are sold together; one bottle containing said first composition and a second bottle containing said second composition.

134. The kit of claim 132, wherein said bottles are sold separately; one bottle containing said first composition and a second bottle containing said second composition.

135. The kit of claim 132, wherein said bottles containing said first composition and said second composition are advertised as more effective if co-administered.

136. The kit of claim 135, wherein said advertisements are selected from one or more of the group consisting of internet, print, and product packaging advertisements.

137. The kit of claim 132 wherein said blister packs are sold together and said blister packs comprise a first blister pack containing said first composition and a second blister pack containing said second composition.

138. The kit of claim 132, wherein said blister pack contains both said first composition and said second composition paired together per unit dose.

139. The kit of claim 132, wherein said blister packs are sold separately and said blister packs comprise a first blister pack containing said first composition and a second blister pack containing said second composition.

140. The kit of claim 132, wherein said blister packs containing said first composition and said second composition are advertised as more effective if co-administered.

141. The kit of claim 140, wherein said advertisements are selected from one or more of the group consisting of internet, print, and product packaging advertisements.

142. The kit of claim 105, wherein said first composition and said second composition are co-administered to a patient.

143. The kit of claim 142, wherein said first composition and said second composition are co-administered to said patient orally.

144. The kit of claim 105, wherein said first composition and said second composition are co-administered to a patient to treat and/or alleviate the occurrence or negative effects of cardiovascular disease.

145. The kit of claim 144, wherein said cardiovascular disease is selected from one or more of the group consisting of congestive heart failure, ischemic heart disease, cardiomyopathy, hypertension, coronary artery disease, high blood pressure, arrhythmia, and thrombogenicity.

146. A method comprising providing the kit of claim 105 to a patients.

147. The method of claim 78, wherein said first composition consists of about 15 mg to about 45 mg vitamin B₆; about 0.9 mg to about 2.7 mg vitamin B₁₂; about 1.25 mg to about 3.75 mg folic acid; and about 50 mg to about 150 mg CoQ₁₀; and said second composition consists of about 50 mg to about 150 mg omega-3 fatty acids.

148. The method of claim 78, wherein said first composition consists of about 30 mg; vitamin B₆; about 1.8 mg vitamin B₁₂; about 2.5 mg folic acid, and about 100 mg CoQ₁₀; and said second composition consists of about 100 mg omega-3 fatty acids.

149. The kit of claim 105, wherein said first composition consists of about 15 mg to about 45 mg vitamin B₆; about 0.9 mg to about 2.7 mg vitamin B₁₂; about 1.25 mg to about 3.75 mg folic acid; and about 50 mg to about 150 mg CoQ₁₀; and said second composition consists of about 50 mg to about 150 mg omega-3 fatty acids.

150. The kit of claim 105, wherein said first composition consists of about 30 mg vitamin B₆; about 1.8 mg vitamin B₁₂; about 2.5 mg folic acid; and about 100 mg CoQ₁₀; and said second composition consists of about 100 mg omega-3 fatty acids.
